(19) United States
(12) Patent Application Publication
(10) Pub. No.: US 2002/0176893 A1
(43) Pub. Date: Nov. 28, 2002

(54) Compositions, implants, methods, and kits for closure of lumen openings, repair of ruptured tissue, and for bulking of tissue

(76) Inventors: John F. Wironen, Alachua, FL (US); Russell S. Donda, Alachua, FL (US)

Correspondence Address: VAN DYKE & ASSOCIATES, P.A., 1630 HILLCREST STREET, ORLANDO, FL, 32803, US

(21) Appl. No.: 10/016,602

(22) Filed: Oct. 22, 2001

Related U.S. Application Data
(63) Continuation-in-part of application No. 09/865,318, filed on May 25, 2001.
Application No. 09/865,318 is a continuation-in-part of application No. 09/776,404, filed on Feb. 2, 2001.

(51) Int. Cl. A61K 9/14

(52) U.S. Cl. 424/489

(57) ABSTRACT

Disclosed and claimed are compositions, devices, methods and kits that are useful in occluding lumens or bulking-up regions of tissues or organs in a living mammal. The invention pertains to compositions, containing specific bioactive components in combination with carriers, and tissue based implants, wherein the bioactive components promote responsive body processes that contribute to the formation of the occlusion or bulked-up region or repair of damaged tissue. Also disclosed is an expandable collagen sponge for implantation into lumens, voids, and cavities.

### CROSS-REFERENCE TO RELATED APPLICATIONS

This application is a continuation-in-part of U.S. application Ser. No. 09/865,318, filed May 25, 2001; which is a continuation-in-part of Ser. No. 09/776/404 filed on Feb. 2, 2001. The benefit of priority is claimed under 35 USC §§119, 120 to foregoing applications, and the teachings of which are incorporated herein in their entirety.

### FIELD OF THE INVENTION

This invention relates to compositions, implants, methods and kits that are useful in minimally invasive surgical procedures, particularly percutaneous applications of a composition that forms a blockage of a lumen of a body tube, repair ruptured tissues, or bulk up existing tissue. Embodiments include the percutaneous injection or insertion of a composition or implant into a lumen, to block the lumen, such as the vas deferens, to effectuate sterilization, or into tissue to repair damage.

### BACKGROUND OF THE INVENTION

The parent application, Ser. No. 09/776/404, which is incorporated by reference, discloses inventions related to the promotion and formation of an adhesion in a living body for a number of treatments and applications. Included in the parent application is the preparation (and associated compositions, devices and kits) of an implant comprising a region having a composition that induces the formation of an adhesion between that region of the implant and a region of a tissue in the living body. One example of the invention is a bladder sling onto which a region of adhesion-inducing particles was adhered. In this and other embodiments such compositions are designed to promote the formation of an adhesion.

The present invention discloses the administration of compositions disclosed in the parent, and other compositions and implants, and related technology, specifically for the closure of body openings, in particular lumens of ducts and vessels, and for the injection of materials that result in adding mass or bulk to specific areas of a tissue or organ. Examples include, but are not limited to: closing the lumen of the vas deferens or Fallopian tubes to effectuate sterilization; blocking entrance to the uterus, injecting compositions to add bulk to a muscle, such as the sphincter muscle of the urethra, to make the muscle more effective in shutting the urethral canal; and injecting compositions into the vocal chords, such as to induce a change in voice.

Regarding sterilization, there are numerous methods of surgically sterilizing a male human or other mammal, in particular, by cutting or blocking the vas deferens through which sperm flow. For instance, U.S. Pat. No. 6,103,254 teaches a method of sterilization in which is delivered to the vas deferens a chemically inert biocompatible synthetic polymer having an equilibrium water content of less than 15%, in combination with a biocompatible solvent and a contrast agent. The polymer precipitate forms in situ in the vas deferens, thereby, according to the claims, sterilizing the subject male mammal. Related applications using the same three components are U.S. Pat. No. 5,989,580 for sterilizing a female mammal, and U.S. Pat. No. 5,667,767 for embolizing blood vessels.

U.S. Pat. No. 6,050,766 teaches a method of embolizing blood vessels in the uterus by accessing through the cervix. An embolizing material is introduced by catheter, through the cervix, into a blood vessel to be embolized, and embolizing material is delivered through the catheter. The embolizing material can include a sclerosing embolic material, a particulate embolic material, and a fluid embolic material. The disclosure states that the procedure may be used to block blood vessels that supply fibroid or tumor tissue.

U.S. Pat. No. 5,826,584 teaches the use of a polymer in a flowable state to be introduced into a channel in a mammal, wherein the polymer, upon cooling to the temperature of the mammal, is non-flowable and blocks the channel. The patent discloses numerous synthetic polymers that melt in the 34 to 45 degree Centigrade temperature range, that are used in the invention. These polymers are generally smooth pastes with particles small enough to pass through channels. However, one common problem with these thermoplastic materials is that they form a physical barrier without interfacing with the surrounding tissue. As a result, these materials may be expulsed from, for example, a uterus through natural uterine contraction and peristalsis.

U.S. Pat. No. 4,920,982 teaches a percutaneous vasectomy method in which a sharp needle is inserted into the vas deferens, which is followed by a blunt needle that provides a cauterizing treatment to the vas deferens. Other methods of sterilization are reviewed in this and the other patents above.

All of the above references are herein incorporated by reference as if each individual reference was specifically and individually indicated to be incorporated by reference in. The references are incorporated to the extent that they are not inconsistent with the teachings herein.

In contrast to the methods and compositions described and claimed in the above patents, the present invention provides methods, compositions and implants that use specific particles in a carrier in the formation of an occlusion or bulked-up region. Particles of preferred embodiments are believed to promote the body's reactions to the particles, which contribute to the occlusion of a lumen in a living mammal, or to the bulking up in a tissue of a mammal.

Other aspects of the present invention are also disclosed which promote an occlusion in a lumen, repair of damaged tissue, or a bulked region in a tissue or organ. Overall, the present invention represents an improvement in the field of implant compositions, surgical materials, and surgery techniques. For the most part, these improvements are mechanistically based on the conception of capturing the benefits of controlled ‘body responsive processes’ which may include the inflammatory reaction and resultant scar or adhesion formation in specific applications, and including cellular infiltration. However, it should be understood that the usefulness of the compositions, devices, methods, implants and kits for induction of adhesions disclosed herein is not to be construed as limited to this, or on any proposed mechanism of the proposed cellular, biochemical and physiological steps thought to be relevant to formation of an occlusion or bulking region.

### BRIEF DESCRIPTION OF THE DRAWINGS

FIG. 1A depicts a normal vessel having an unoccluded lumen.

FIG. 1B depicts injection of a material into a lumen which results in an immune response.

FIG. 1C depicts a vessel having an occluded lumen.

FIG. 1D depicts blockage of material due to occlusion and the formation of a secondary adhesion.

FIG. 2A depicts injection of a biomaterial into a lumen of a vessel which causes tissue damage.

FIG. 2B depicts an immune response in a vessel resulting in scar tissue formation.

FIG. 2C depicts a complete occlusion of a vessel by scar tissue, and the resultant blockage of material flow through the vessel.

FIG. 3A depicts a cross section of a tissue comprising epidermis, dermis and subdermis layers and associated cells.

FIG. 3B depicts injection of a biomaterial into the dermis layer of tissue which causes An immune response in surrounding tissue.

FIG. 3C depicts swelling of tissue resulting from immune response to injection of material.

FIG. 4A depicts a cross section of tissue comprising epidermis, dermis and subdermis layers and associated cells.

FIG. 4B depicts injection of a biomaterial into the dermis layer of a tissue that causes damages to surrounding tissue.

FIG. 4C depicts an immune response to injection and the production of scar tissue.

FIG. 4D depicts swelling in tissue associated with immune response and resulting scar tissue formation.

FIG. 5A depicts an expandable collagen based sponge for insertion into a uterus.

FIG. 5B depicts a reduced expandable collagen based sponge for insertion into a uterus after dehydration and folding.

FIG. 5C depicts a reduced expandable collagen based sponge and an implant syringe.

FIG. 5D depicts a reduced, expandable collagen based sponge inside an implant syringe.

FIG. 6A depicts an implant syringe containing a reduced expandable collagen based sponge, wherein the implant syringe is inserted into the uterine cavity

FIG. 6B depicts an implant syringe containing a reduced expandable collagen based sponge, wherein the sponge is forced through the syringe and into the uterine cavity.

FIG. 6C depicts a collagen based sponge inserted into the uterine cavity in an expanded form to block access to the uterine passageway

FIG. 7A depicts an intervertebral disc complex showing a ruptured disc.

FIG. 7B depicts an injection of bioactive material into the damaged area to repair the annulus fibrosus.

FIG. 7C depicts an intervertebral disc complex having a repaired disc.

### SUMMARY OF THE INVENTION

Regarding the closure of lumens of tubes or vessels in the body, compositions, and administration, including percutaneous administration, of a composition or object into a lumen are disclosed and claimed. Specific embodiments include the closing of the vas deferens in a male, to effectuate sterilization. Another embodiment regards the lessening or cessation of menstrual flow, or menorrhea, in females who experience heavy flow through reproduction of the symptoms of Asherman's syndrome. In another embodiment, an injection of bioactive composition at a site of intervertebral disc rupture is used to repair a torn annulus fibrosus. Also included in the scope of this invention are the closures of other ducts, tubes, or other channels having a lumen of which closure is desired. Examples include, but are not limited to, the lumens of tear ducts and arteries (such as in arteriovenous anastomosis).

Other applications include using the compositions to increase bulk in order to increase the competency of sphincter muscles located throughout the body. This involves injection or other administration of a composition directly into the sphincter muscles. See U.S. Pat. No. 5,490,984, where this approach using collagen has been shown to alleviate anorectal and/or urinary incontinence. This increase in muscle bulk counteracts the stretched condition of a muscle, tightening it, and thereby aiding in the treatment of an individual having incontinence problems due to a weakened or stretched muscle of the urethra. Also, injecting such a composition into vocal chords bulk up this area, leading to a change in voice characteristics.

Thus, one important objective of the invention is to provide a method of blocking a lumen, or channel, in a living mammal. Another important objective is the provision of the compositions or implants that are used in this method to effectuate such blocking.

Another objective is to provide a method that provides a blocking or bulking composition to a target lumen or tissue area, respectively, which includes the administration of the composition percutaneously, as for example with a syringe.

Another object of the present invention is to provide a method and compositions for the repair of a ruptured intervertebral disc.

Another objective is to provide kits that include necessary components and convenient, reliable devices for the utilization of the invention.

An advantage of the invention is the use of largely natural materials and processes to form a blockage or bulking area, thereby avoiding the use of potentially toxic or reactive synthetic blocking materials.

Another advantage of the invention is that the variation in the size of the lumen from one application to another is well accommodated by the variable filling amount allowed by the method, avoiding the need to match sizes if one were using different pre-formed plugs.

The subject injectable compositions may be thermoplastic, i.e., capable of flowing at temperatures somewhat elevated above typical mammalian body temperatures, but are solid or non-flowing at such body temperatures, however thermoplasticity is not preferred.

According to another embodiment, the present invention is directed to an implant that is highly compressible when dehydrated, i.e., capable of being folded in upon itself to significantly reduce its size such that it may be placed into the barrel of a syringe for injection. This allows an implant which, upon injection into a lumen of other body cavity, will expand to its original size when rehydrated, and thereby effectively block the lumen or channel.

Additional objects, advantages, and novel features of the invention will be set forth in part in the description which follows, and in part will become apparent to those skilled in the art upon examination of the following, or may be learned by practice of the invention. The objects and advantages of the invention may be realized and attained by means of the instrumentalities and combinations particularly pointed out in the appended claims.

### DETAILED DESCRIPTION OF THE INVENTION

The foregoing and other objects and advantages are attained by a variety of compositions, devices methods, and kits that promote the occlusion of a lumen in a living body of a mammal, or that add bulk to a tissue of a mammal, for a variety of purposes and applications.

The invention provides methods for completely or partially blocking, sealing, filling, or adding bulk to various lumens or regions of muscle or tissue within the body of a patient. As used herein, the term “lumen” is intended to encompass the space within various hollow organs or vessels of the body, such as the vas deferens, Fallopian tubes, veins, arteries, intestines, trachea, uterus, and the like. As used herein, the term “closure” is intended to mean the complete or partial blockage, sealing or occlusion, of a space, such as a lumen or channel, which thereby impairs or blocks passage of material through the space.

In an alternative embodiment, the subject methods are useful for a form of birth control or sterility in females, wherein the biomaterial is injected, or implanted, such that the Fallopian tubes are filled or blocked by the biomaterial, thereby preventing egg and/or sperm from passing through or around the biomaterial. Using this approach, pregnancy would be prevented since the ova or eggs located in the Fallopian tubes would not exit to the uterus and would not make contact with sperm. The blockage, and hence the sterility or birth control, is reversible by removal of the biomaterial or re-sectioning of the tube after surgery, wherein the blocked portion of the tube is excised and the remaining portions of the tube are reconnected. It is preferable that the sections of the Fallopian tubes blocked with the biomaterial are those directly connected or closest to the uterus. Administration of the biomaterial for this therapeutic indication can occur via catheter or via endoscopes, such as a fiberoptic scope, hysteroscope, and the like. See “Hysteroscopic Approaches for Tubal Closures,” John J. Sciarra, Research Frontiers in Fertility Regulation, 1980, Chapter 26, pp. 270-286. Preferably, the biomaterial is injected into the Fallopian tubes using a catheter.

The administration of the biomaterial via implant or injection is minimally invasive and usually can be performed on an outpatient basis, resulting in a lower cost than other surgical forms of sterility or birth control. The procedure also eliminates issues of patient compliance, since the patient need not follow any specific instructions or remember to ingest or insert other forms of birth control, such as pills, diaphragms, and the like. However, supplemental forms of birth control can be utilized, if desired, especially those which prevent disease transmission.

According to the most general method of the invention, an effective amount of a biomaterial composition is administered to the site of a lumen or void within the body of a patient. The term “effective amount”, as used herein, means the quantity of biomaterial needed to augment, block, or fill the biological structure of interest. The effective amount of biomaterial administered to a particular patient will vary depending upon a number of factors, including: the sex, weight, age, and general health of the patient; the patient's own ability to absorb or break down the biomaterial; the type, concentration, and consistency of the biomaterial; and the particular site and condition being treated. The biomaterial may be administered over a number of treatment sessions.

As described above, an effective amount of one or more biologically active agent, such as a wound healing agent, antibiotic, or antimicrobial agent, can be incorporated into the biomaterial composition. In this context, an “effective amount” refers to the amount of biologically active agent, antibiotic, or antimicrobial agent required to obtain the desired therapeutic effect, such as improved or accelerated healing of the defect or void, or prevention of infection at the site of administration.

“Biologically active agent” as used herein includes, but is not limited to, antiviricides, particularly those effective against viruses such as HIV and hepatitis; nonoxynol-9; chlorhexidine; benzalkonium chloride; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin, etc.; amino acids, magainins, peptides, vitamins, inorganic elements, co-factors for protein synthesis; hormones; endocrine tissue or tissue fragments; enzymes such as collagenase, peptidases, oxidases, etc.; polymer cell scaffolds with parenchymal or other cells; surface cell antigen eliminators; angiogenic or angiostatic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, growth factors, growth hormones such as somatotropin; bone digestors; antitumor agents; glycosaminoglycans, proteoglycans, fibronectin; cellular attractants and attachment agents; immuno-suppressants; adjuvants such as Freunds complete adjuvant, lipopoylcaccharides, vegetable oil, etc.; permeation enhancers, e.g., fatty acid esters such as laureate, myristate and stearate monoesters of polyethylene glycol, enamine derivatives, alpha-keto aldehydes, etc.; nucleic acids; bioerodable polymers such as those disclosed in U.S. Pat. Nos. 4,764,364 and 4,765,973, and combinations of any of the foregoing. The amounts of such medically useful substances can vary widely with optimum levels being readily determined in a specific case by routine experimentation.

The term “growth factor” as used herein refers to a polynucleotide molecule, polypeptide molecule, or other related chemical agent that is capable of effectuating differentiation or proliferation of cells. Examples of growth factors as contemplated for use in accord with the teachings herein include a epidermal growth factor (EGF), transforming growth factor-alpha (TGF-alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), and/or platelet derived growth factor (PDGF).

As used herein, the term “effective amount”, whether in reference to a biomaterial or biologically active agent, also refers to that amount of material which is pharmaceutically and physiologically acceptable to the particular patient undergoing treatment.

Furthermore, the subject methods can be used to treat incontinence due to incompetent sphincter muscles along the GI and urinary tracts. Treatment involves the injection of the subject compositions directly into the sphincter muscles.

Apart from the novel thermoplastic characteristics of some of the compositions, the composition can be further localized by the use of a clamp, balloon catheter, umbrella, surgical instrument, and the like. Injection of a biomaterial between a dual balloon catheter can be used to block the lumen anterior and posterior to the catheter tip.

One method for removal of an occlusion, if desired, is the in vivo degradation of the composition, for example, by enzymes such as collagenase. The rate of degradation in vivo and eventual resorption by the body can be controlled by varying a number of factors including, without limitation, the type and concentration of the collagen or other carrier used. This removal process also would need to remove the cellular growth and extracellular molecular structure that was formed in response to the administered composition. Accordingly, a combination of degradative enzymes may be used to effectuate such removal.

Summary of Compositions:

One class of preparations, or compositions, that are injected to form obstructions include at least one water-insoluble material known to promote responsive body processes, including cellular infiltration, and a carrier having the property of suspending the material(s) in suspension. In the first step, one or more materials are combined with the carrier. Once well mixed, this preparation is ready for administration to a lumen or area for bulking or sealing. When placed in a patient during a percutaneous or other means of delivery, each specific area so treated has the capability to inducte or promote the formation of an occlusion of the lumen, or bulking-up of the tissue or organ area with which the preparation is in contact.

For the compositions and the methods of the present invention, the water-insoluble material may be selected from or may be a combination of materials selected from the following non-exclusive list: fine particles of bone; fine particles of hydroxyapatite, preferably in the 1 to 70 micrometers particle size range; non-osteoinductive precipitated demineralized bone matrix (DBM), collagen particles (prepared and sized without focusing on obtaining sharp-edged particles), preferably in the 1 to 70 micrometer particle size range; collagen shards (prepared and sized to preferentially obtain sharp-edged particles), preferably in the 1 to 70 micrometer particle size range; insoluble salts, synthetically derived compounds found in bone (e.g., synthetic hydroxyapatite), and talc. Cross-linked tissues, such as with glutaraldehyde, may also be used for this purpose. Required characteristics of such material are: non-toxic or mildly toxic; insoluble in water; and capable of inducing a mild inflammatory response to induce fibrosis. Preferred characteristics are that the material is biodegradable and will disappear in between 7 and 30 days of introduction into the person or animal or after formation of a stable granuloma.

Additionally, it is preferred that the starting materials are processed in such a way as to yield particles that predominately are needle-shaped with sharp, or pointed, and not rounded, edges. Such parts may be obtained by fracturing the starting materials. While not wishing to be bound by a particular theory, it is believed that such sharp-edged particles are more effective at irritating and inflaming tissue to form an adhesion. A preferred embodiment of the composition has over 50 percent of the particles having, on microscopic inspection, at least one side or end with an angle less than 40 degrees, and preferably less than 30 degrees. A group of such particles is termed “predominantly sharp-edged” for the purposes of this disclosure.

The preferred source of the bone, bone derivatives, and collagen is from human tissue, such as cadaverous tissue. Other possible sources are bovine, ovine, and other non-human cultured species, and synthetic sources, e.g., synthetic hydroxyapatite. Combinations of these materials are also contemplated. Further, those skilled in the art will appreciate that a vast array of materials may meet this requirement and hence may be used according to this invention, even though not specifically mentioned herein.

It is noted, that throughout this specification, including the claims, by “particle size range” is meant that the median of the range of particle sizes for the middle 80 percent of the total particle mass falls within the specified range. That is to say, excluding the 10 percent of the smallest particles, and the 10 percent of the largest particles, on volume, number or mass basis, the median of the remaining smallest and largest particles falls within the specified numerical range. Thus, a 1 to 70 micrometers particle size range may include some particles smaller than 1-micrometer in maximum dimension, and some particles greater than 70 micrometers in maximum dimension, but the median of the majority of the particles falls within the 1 to 70 micrometers range.

It is further noted that the size distribution, general shape and sharpness of the particles of materials, and the concentration of such particles in the carrier, is adjustable for the intended application. Thus, where a rapid body response is desired, a higher level of particles with pointed or sharp ends may be employed. The proper combinations can be readily determined by routine evaluations by one of ordinary skill in the art given the type or particles to be used and the particular application.

An alternative composition is specially processed dermis that has increased retention in the body, while possessing a decreased rate of rejection. This is termed acellular dermis.

Other biologic and synthetic materials may be used to aid adhesion between tissues. For example, one or more natural or synthetic glycosaminoglycans (mucopolysaccharides), such as, for example, hyaluronic acid (HA), chondroitin sulfate, dermatan sulfate, keratin sulfate, heparin, heparin sulfate, galactosaminoglycuronglycan sulfate (GGGS), and others, including their physiological salts, may be used alone or as a carrier, or a cross-linked form of the foregoing. These substances have been implicated in cell-cell, or cell-matrix adhesion activities. Hyaluronic acid (HA), for example, is a matrix component that has been associated with many different cellular processes including cell adhesion. Proteoglycans, such as, for example, heparin sulfate proteoglycan (HSPG) are also involved in regulating cell-cell adhesion and may be injected to cause an occlusion between tissues. Additionally, fibrin glues or sealants may also be used. Other adhesion proteins, such as, for example, laminin, entactin, nitogen, as well as members of the selectin, IgSF, integrin, and cadhedrin superfamilies may be used alone or in combination to create an adhesion in a target tissue.

Where it is desired to add bulk to a tissue or organ, a number of natural or synthetic materials may be used. High molecular weight HA, for example, may be injected percutaneously to add temporary bulk to a tissue. Proteoglycans, particularly aggrecan, play an important physiochemical role in the maintenance of disc hydration and morphology and may also be injected directly into a tissue to increase mass. Other natural allogenic, autogenic or xenogenic materials capable of providing mass to a tissue also fall within the scope of this disclosure as possible injectables. For example, Zyderm™ (suspension of bovine collagen fibrils) and Zyplast™ (suspension of bovine collagen fibers crosslinked with glutaraldehyde) may be injected percutaneously to add bulk to a tissue. Fibrel™ (mixture of plasma, porcine derived gelatin, and e-aminocaproic acid) is also a good candidate for injection because it has been shown to induce collagen formation in situ. Homologous materials, such as, for example, Dermalogen™ (homologous collagen dispersions containing collagen, chondroitin sulfate and other proteoglycans) are also contemplated for injection into a tissue due to its ability to promote cartilage production. Further, an injectable form of Micronized Alloderm™ (acellular matrix of human dermal proteins and proteoglycans) may also be injected into a tissue to add bulk. Autologous materials such as Isologen™ (cultured autologous fibroblasts capable of producing collagen upon injection) are also contemplated herein for injection to add tissue mass. Further, phospholipids may be injected where temporary biomass increase is desired.

Use of synthetic materials to add bulk to a tissue is also contemplated. Examples of injectable synthetic materials that may be used include medical grade silicone, Bioplastique™ (solid silicone particles suspended in polyvinylpyrrolidone carrier), Arteplast™ (microspheres of polymethylmethacrylate (PMMA) suspended in gelatin carrier), Artecoll™ (smooth PMMA spheres suspended in bovine cartilage carrier). Further, synthetic hydrogel compositions may be used as a filler material to increase tissue mass. Formacryl®, for example, is polyacrylamide (5%), a synthetic polymer, suspended within apyrogenous water (95%). Following precutaneous injection, water is absorbed by the body, and the remaining material remains soft and pliable making it an ideal choice for situations where long term use is indicated. Bioplastique™, may also be used as a filler. Materials such as expanded polytetrafluorethylen (e-PTFE), CoSeal™ (synthetic hydrogel) and others may be used alone or as a carrier fluid for a variety of materials that have been described herein.

For the devices and the methods of the present invention, the carrier may be selected from or may be a combination of materials selected from the following non-exclusive list: collagen; gelatin; carboxymethyl cellulose; glycosaminiglycans; proteoglycans, hydrogels, polyvinyl alcohol; thrombin; fibrin; albumin; and mucoadhesive polysaccharides such as chitosan, polyalcohols, polyamines, polyvinyls, polyamides, polyesters, polyanhydrides, polyortho-esters, polyurethanes, polycarbonates, polyphosphazines, and polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and similar materials suitable for use as a carrier. Also, the composition may contain, for example in the carrier, growth factors including but not limited to PDGF, FGF, VEGF, BMP, and/or biologically active agents. Required characteristics of the carrier are: non-toxic; able to suspend the water-insoluble particles during application onto an implant; and retains a functional shape once delivered in the lumen or target tissue or organ.

A preferred embodiment of the present invention is a specific composition that promotes the formation of an occlusion by promoting responsive body processes, including a localized inflammatory response. This composition comprises hydroxyapatite particles in the size range of 1 to 1,000 micrometers, generally having sharp points. For formation of a closure of a lumen, the composition, including the hydroxyapatite particles, should degrade within 10 years, and preferably degrades between 7 and 30 days after implantation. However, for bulking up of tissues, such degradation is not necessary, and is undesirable. For such bulking up applications, highly crystalline hydroxyapatite is preferred. Thus, the selection of the type of water-insoluble particle will depend on a particular application and objective.

In one embodiment, the selected particles are suspended in a carrier, such as, but not limited to, gelatin. Typically the gelatin is made into an aqueous solution having a 1 to 70 percent gelatin weight/total weight of aqueous solution (before adding particles), preferably 5 to 40 percent weight/total weight in aqueous solution. The volume of unpacked hydroxyapatite particles which are added to a volume of this solution should comprise approximately 50 to 100 percent of the total implant volume (e.g., the total volume of the combined gelatin aqueous solution, particulate hydroxyapatite, and other components of the implant composition). These particles are mixed into the solution to form a slurry or suspension in which the carrier solution (e.g., the gelatin solution) occupies spaces between the particles, and, depending on the relative concentrations, may fill a larger space between the particles.

This composition may be utilized for application to areas to cause or promote closures or occluding of lumens, or bulking or sealing of tissues. For example, one specific surgical procedure to benefit from the present invention is a vasectomy. In one example, an injection of a composition of the present invention is injected into a small area of the vas deferens, resulting in a blockage effectuating sterilization. Another example is blockage of arteriovenous malformations, as by injection of a composition according to the present invention. Other applications include the blockage of tear ducts, salivary gland ducts, and sweat gland ducts, and the bulking up of sphincter muscles and vocal chords.

While not wishing to be bound to a particular theory, it is believed that the possible mechanisms for the formation of an occlusion in a lumen, and the bulking of tissue upon administration of the composition according to this invention, is one or more of the following: inflammation leading to scar tissue formation and/or adhesion; cellular infiltration in which fibrous or other tissue grows over particles of the composition, and other cellular, chemical, biochemical, and/or physiological reactions upon the mass that modify the mass while the functional obstruction of the lumen is maintained. These possible mechanisms are collectively referred to in this application as ‘responsive body processes’ and are not meant to be limiting as to other general or specific processes that may be initiated as a result of the implant.

It is noted that the induction or promotion of such responsive body processes by particles in the composition of the present invention is believed to be one factor that differentiates the present invention from other methods and compositions in the art that use largely synthetic polymers or adhesives to form a plug or seal tissues together. While such synthetic materials may promote some superficial reactions, their compositions remain largely intact, and are not acted upon by body processes. In contrast, the compositions of the present invention may be largely acted upon, and/or resorbed over time, creating a different type of occlusion or bulked-up region.

Thus one general embodiment of the present invention is a method of forming an occlusion or bulked-up area by the steps of:

1. preparing a preparation of particles that, upon placement in a living body, promotes responsive body processes, and

2. injecting a quantity of said composition into a lumen, tissue, or organ.

The injection promotes formation of an occlusion or bulk-up region in the area of the injecting. Multiple injections may be given at one time, in different body areas.

A further embodiment is to prepare the particles in an aqueous solution of gelatin, to form a suspension for injecting. A further embodiment is to inject the preparation using a syringe, such as via a percutaneous injection. Yet another embodiment is to inject a tissue based implant that forms an occlusion by expanding to block a passageway.

Thus, one advantage of some embodiments of the present invention over other methods that use synthetic polymer compositions that may set rapidly upon administration, is that the compositions of the present invention do not tend to stick to the end of the needle, syringe or other applicator during administration (see, in contrast, U.S. Pat. No. 5,826,584, column 3, line 46). In the present invention, a sufficient quantity of material may be injected as a bolus, expanding the lumen and effectively closing it off. During a transition including, for example, cellular infiltration to absorb the particles in the bolus, the composition maintains the occlusion. The occlusion is made more permanent with, for example, ongoing cellular infiltration and other cellular and biochemical/immunological processes upon the composition.

Depending on the site of injection, this method may result in the narrowing or the closure of a body opening. For instance, where it is medically desirable or necessary to close the cervical opening of the uterus, an injection of the preparation at or near the cervical opening results in adhesion formation (also known as scar tissue or granulation tissue formation) that closes the uterus. More broadly, this method can be applied to a wider range of medical conditions where it is desirable to close or narrow an opening.

It is noted that, depending on the nature of the surgery and the anatomy and condition of the patient, certain procedures may be used to assure desired positioning of the composition, at least until an adhesion or occlusion is formed. For instance, the application of one or more enzymes that promote a binding of adjacent structures, such as by enzymatic catalysis, may be used to maintain the desired juxtaposition during formation of an adhesion. Specifically, U.S. Pat. No. 5,549,904 (Juergensen et al.) is incorporated by reference regarding a biological adhesive composition employing as the key ingredient a tissue transglutaminase enzyme. This enzyme has been found to promote adhesion between tissue surfaces by catalyzing the reaction between glutaminyl residues and amine donors. Such technology is applied by incorporating into the composition, or preparation, glutaminyl residues and/or amine donors, and materials providing a solution of the enzyme on the lumen cell surface, or other target area, prior to administering the composition or preparation.

Examples of Applications

It will be appreciated by those skilled in the art that the specifics of the composition of this invention, its method of preparation and use are applicable to such compositions for use in any mammal species. Nonetheless, because human use is considered likely to be the principal application of this new material, the following description concentrates on exemplifying this material for human applications.

In one embodiment, pulverized dermis having a particle size range of approximately 40-120 micrometers was added to a 5 percent solution of gelatin to reach a 90 percent volume of dermis particles to total volume of prepared implant material. The gelatin solution is liquid at temperatures above approximately 20 degrees Centigrade. However, because the relative solids concentration is so high, the final implant consistency, namely ranging between a slurry and a paste, is such that the implant composition can be applied for the purposes of this invention.

In a second embodiment, a composition is prepared by mixing allograft-derived bone particles that have been sized to pass through a 20-200 micrometer mesh screen and that remain on a 5-50 micrometer mesh screen. These particles are obtained by separation on a gyrating laboratory sieve. 1.5 cubic centimeters of these unpacked particles are suspended by mixing by stir bar, until homogeneous, into a three milliliter volume of a 20 to 30 percent gelatin solution, the preparation of which is described in detail below. This suspension is largely stored in appropriate sterile vessels and stored frozen. As needed, a 0.25 to 0.50 ml. of this suspension is loaded into a sterile syringe. The tip is sealed with a sterile cover and warmed in a water bath at 40-45 degrees Centigrade.

A male subject in need of sterilization is then prepared for the procedure. One of the vas deferens is manually located and held between the thumb and index finger of the surgeon or assistant. A local anesthetic is applied to the area above the planned site of injection, and also the vas deferens itself is infiltrated with anesthetic. The other vas deferens is similarly infiltrated.

Then a specialized vas deferens fixing clamp (see U.S. Pat. No. 4,920,982, for an example) is applied over the scrotum skin and underlying vas deferens, and closed to tighten around the vas deferens. The needle of the syringe containing the composition then pierces the scrotum skin and its tip enters the lumen of the vas deferens. Once position is confirmed, a quantity of the composition is injected into the lumen of the vas deferens. Optionally, the injection continues until a slight lump is visually observed in the vas deferens, representing the mass of the composition. The needle is then withdrawn and normal postoperative procedure is followed for treating the puncture. The other vas deferens is similarly occluded.

The same composition may also be applied to effectuate sterilization of a female. After appropriate preparation and anesthetization, as needed, a catheter attached to a syringe is directed to each Fallopian tube, and an effective amount of the composition is injected into each tube to occlude the tube. Compositions are prepared within the parameters according to this invention, and are applied in this or other methods of application known to those skilled in the art, to occlude each Fallopian tube to effectuate sterilization.

Another embodiment regards the lessening or cessation of menstrual flow, or menorrhea, in females who experience heavy flow. One embodiment is to apply a composition of the present invention to the uterus of a female in need of lessening or cessation of menstrual flow. Responsive body processes result in the partial or complete functional loss of the endometrium lining, resulting in the desired lessening or cessation of menstrual flow. The preferred application of such composition is by catheter through the cervix. Monitoring of the application of the composition can be done by hysteroscope or other means known in the art.

Alternatively, a tissue based implant material may be formed for implantation into the uterus to simulate symptoms in a uterus associated with Asherman's syndrome. In a preferred embodiment, a collagen gel is cross-linked and solidified to form a sponge, with or without the combination of sharp shards. The sponge is then dried and compressed to form a cylinder and delivered via injection, catheter, or similar route into a uterus. Upon rehydration, the implant expands to fill and effectively block the uterine opening. Unlike other temporary implants such as, for example a diaphragm, retention of the implant is achieved through absorption of blood to form a clot that will hold the implant in place. Implantation causes adhesions in the uterus similar to those observed in Asherman's syndrome in women with benign ammennorhea. These adhesions block sperm migration up into the uterus, prohibiting an embryo from implanting into the uterine lining, which results in infertility. Preferably, a DNC is performed prior to implantation.

In an alternative embodiment, compositions may be used to repair fissures within an annulus fibrosus or to seal openings made during removal of a damaged nucleus pulposus. Disc abnormalities may be the result localized tears or fissures in the annulus fibrosus resulting from the trauma, or the natural aging process. Structural degeneration of fibrous components of the annulus fibrosus increase with age often resulting in tears from disc herniations. Traditionally, repair of tears to the annulus fibrosus included bed rest, pain medication, physical therapy or steroid injection. Alternative methods of sealing ruptures to the annulus fibrosus include application of various forms of cauterization, including electromagnetic radiation, and electromyographic techniques to cause the tissue to seal. More recently, patents have issued on methods to seal ruptures of the annulus fibrosus. U.S. Pat. No. 6,290,715 issued to Oratec Interventions, Inc Sep. 18, 2001 discloses a method of inserting a heating element into a disc to cause collagen of the annulus fibrosus to weld together thereby forming a seal. U.S. Pat. No. 6,224,630 issued to Advanced Bio Surfaces, Inc, May 1, 2001 discloses an implantable tissue repair device adapted to be sealably positioned within the annulus of an intervertebral disc. None of these methods utilize a bioactive agent capable of repairing tears in the annulus fibrosus through interaction of natural body reposne processes described above.

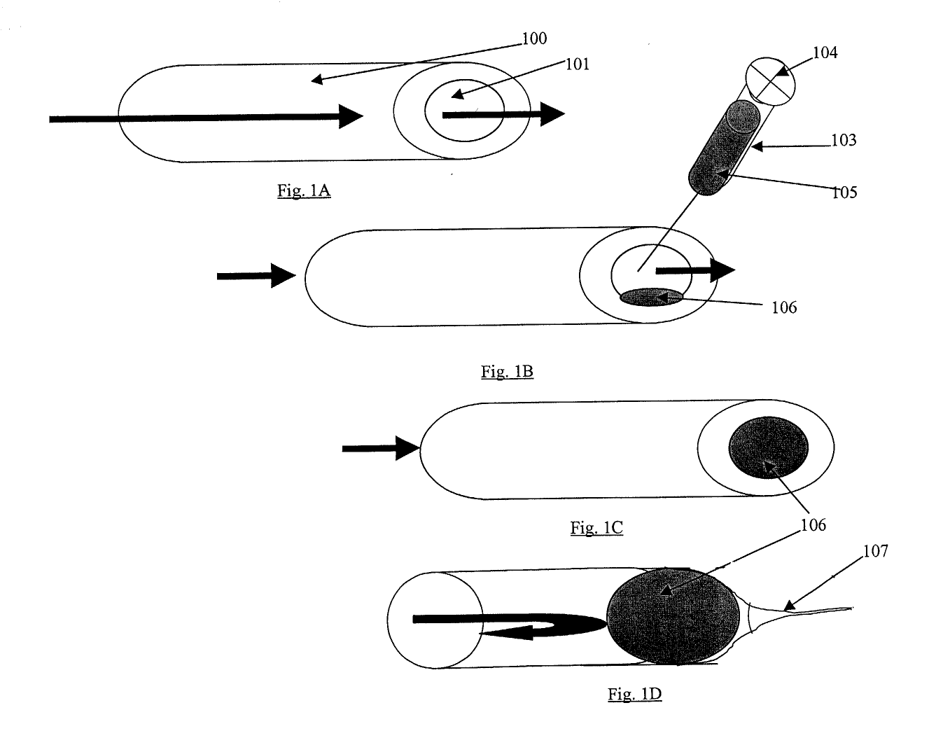

The methods used to form an occlusion or to bulk up tissue as described above are shown in FIGS. 1-7. FIG. 1A shows a normal vessel 100 having an unobstructed lumen 101 with a general material flow in the direction of the arrow. FIG. 1B depicts a percutaneous injection via a syringe, 110, having a plunger, 111, and an attached hollow needle 112, and containing a flowable composition, 120. The end of the needle, 112, is positioned in the lumen, 101, of a desired vessel. After confirming proper positioning, as by radiographic or visual means, a desired quantity of the composition 120 is injected into the lumen 101. Once injected, an immune response begins via signaling action of surrounding native cells 102 which begin to migrate toward the site of injection. FIG. 1C shows a portion of a vessel having the lumen filled with the composition 120, thereby forming an occlusion 130 at the site of injection. Native cells 102 release materials 103 in response to the foreign matter introduced via injection into the lumen, which adds to the swelling. The needle is then withdrawn, and a dressing, as needed, is placed on the external puncture caused by the needle. In a preferred embodiment, the composition 120 is flowable at an elevated temperature, and solidifies upon equilibration with the temperature of the subject living mammal. FIG. 1D shows a vessel 100 having an occlusion 130 that blocks the forward flow of material passing therethrough, as depicted by the arrow. Depending upon the vessel architecture, a portion of the vessel forward of the occlusion may collapse in on itself to form a secondary natural adhesion 140 of tissue. Once injected the material forms an occlusion that prevents material movement through that section of the vessel, thereby effectuating sterilization, birth control or other desirable results. In this application, the occlusion need not be permanent, and may be removed by dissolving or otherwise breaking up the material forming the blockage. In situations whereas a more permanent blockage is desired, a composition may be injected which is capable of promoting a bodily response that will result in scar or other tissue formation within the vessel to create a blockage. In an alternative embodiment, arterial-venous malformations are blocked by grinding cross-linked collagen sponge into particles 20-40 microns across, compressing them, and delivering them in a non-solvent to the collagen (e.g. ethanol, hypertonic saline, etc.).

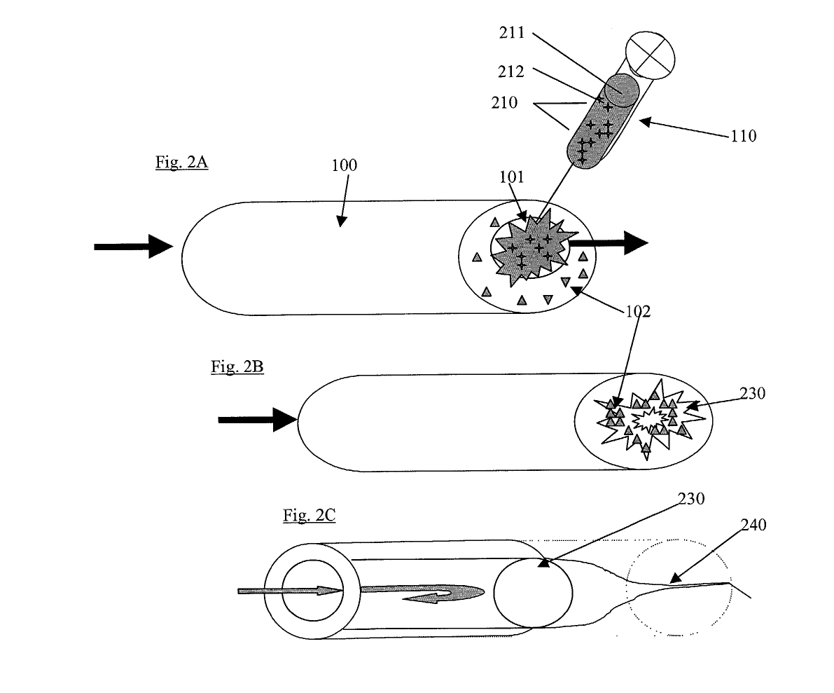

FIGS. 2A-C depict an occlusion formed through promotion of scar tissue production. FIG. 2A depicts a vessel, wherein a biomaterial 210 comprising a carrier 211 and a bioactive agent 212, is injected into the lumen 101 via a syringe 110. Once injected the biomaterial disrupts the interior surface of the vessel to cause an inflammatory reaction. Native cells 102 respond to the site of injury. FIG. 2B depicts the infiltration of native cells 102 into the site of injection. As the native cells 102 being to repair damage, scar tissue 230 forms in the area, thereby increasing tissue mass and further narrowing the lumen. FIG. 2C depicts a mass of scar tissue 230 formed to repair the site of damage. The scar tissue forms a natural occlusion which effectively blocks material flow through the vessel as depicted by the arrow. Over time, the portion of the vessel forward of the occlusion may collapse upon itself and form a further adhesion 240 to completely seal off the end of the vessel.

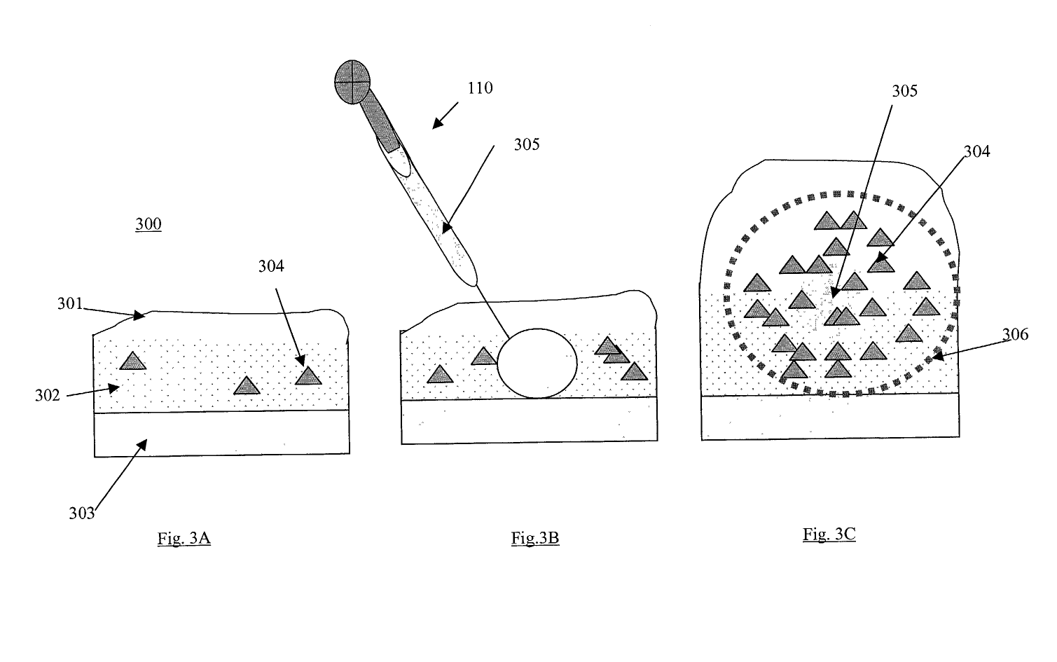

FIGS. 3A-C depicts a percutaneous injection of material to bulk up a tissue. FIG. 3A shows a typical skin layer generally indicated at 300 comprising the epidermal layer 301, dermal layer 302 and subdermal layer 303 and associated native cells 304. FIG. 3B shows injection via a syringe 110 of a material 305 to increase the mass of the tissue. An effective amount of material 305 is injected into the dermal layer to cause a desired degree of swelling. This swelling causes an immune reaction which, in turn, causes native cells 304 to migrate into the area. FIG. 3C depicts a post injection reaction where an amount of material 305 sufficient to cause an immune response causes significant numbers of native cells 304 to migrate to the area. In response to this injected material, native cells 304 release fluid materials 306 to surround and destroy the foreign material which, in turn, promotes swelling and raises the tissue level. Because the swelling does not significantly involve the production of scar tissue, the swelling is of a temporary nature and may be reduced through dissolution of the injected material.

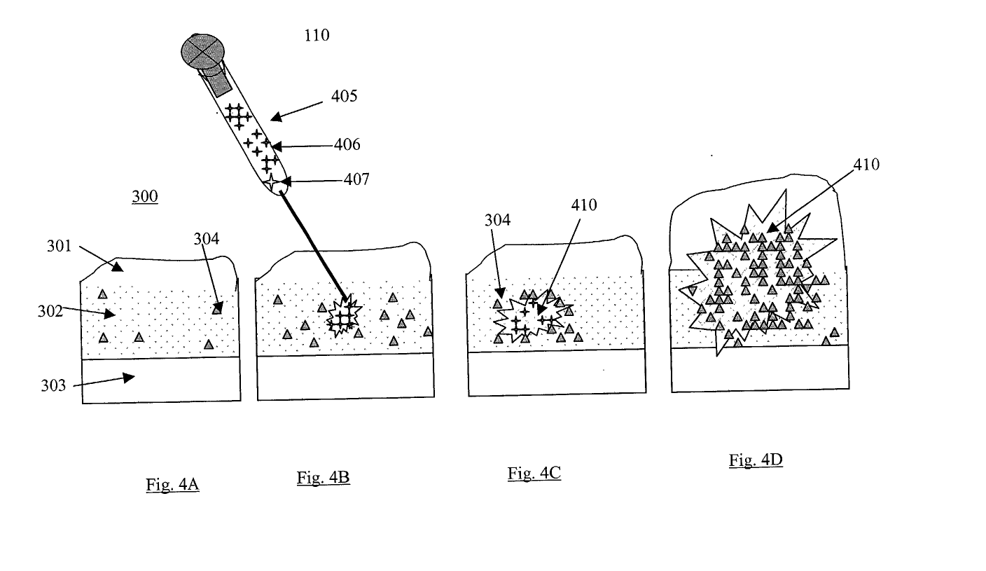

FIGS. 4A-D depict a method of permanently bulking up a tissue. FIG. 4A shows a typical section of tissue indicated at 300 comprising the epidermal layer 301, dermal layer 302 and the subdermal layer 303 and associated native cells 304. FIG. 4B depicts a biomaterial 405 comprising a carrier material 406 and a bioactive agent 407 being injected into the dermal layer of the tissue 300 via a syringe 110. Upon injection, the biomaterial causes damage to adjacent tissue, prompting an immune response by native cells 304. FIG. 4C depicts the activity of native cells 304 to repair the site of damage resulting in the production of scar tissue 410. As repair continues, scar tissue 410 production increases near the site of injection. FIG. 4D shows a mass of scar tissue 410 produced during repair that result in a natural, permanent increase in tissue mass.

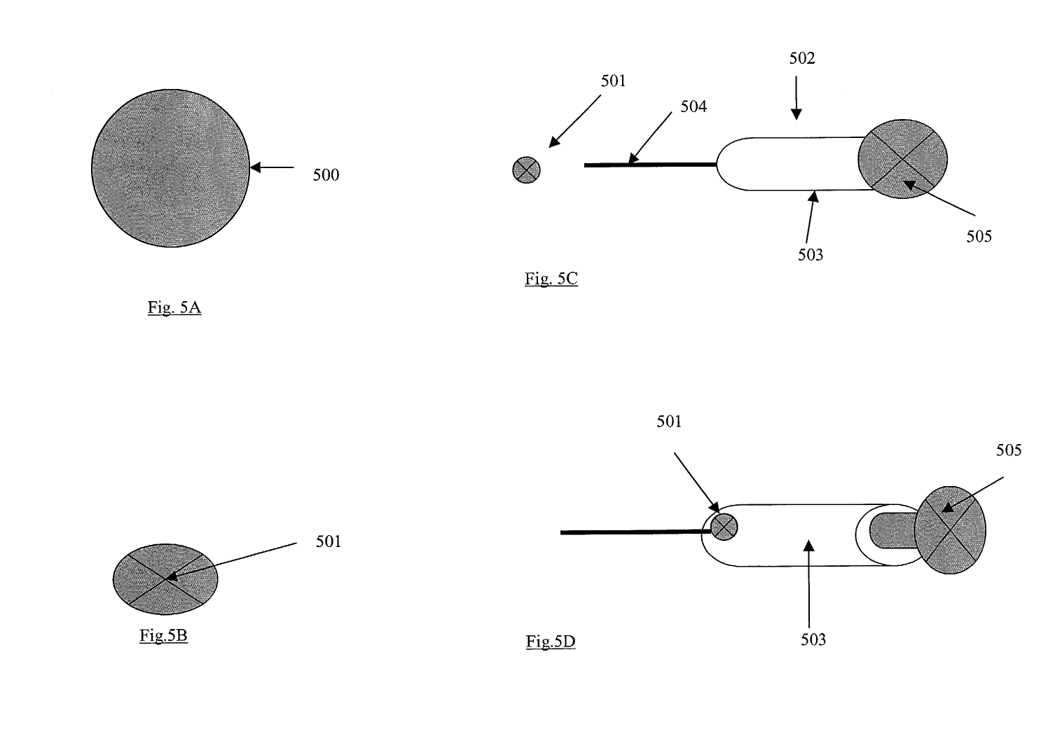

FIGS. 5A, B, C and D depict an expandable collagen based sponge suitable for inducing surgical adhesions or other tissue formation. The device works similar to a tampon. Collagen gel loaded with an agent to induce adhesions can be solidified into a sponge through a number of processes. The sponge is sufficiently cross-linked, dried and then compressed into a much smaller implant that expands to its original size when rehydrated in situ. The implant enters through the cervix or other entry hole and then expands such that it cannot be easily forced back out the entry hole. This overcomes prior issues of implant expulsion through peristaltic action. Further retention of the implant is achieved through absorption of blood to form a clot that will hold the implant in place. Induction of adhesions in the uterus reproduces Asherman's syndrome in women with benign mennorhea. FIG. 5A depicts an expandable collagen based sponge 500 according to the present invention. FIG. 5B depicts the expandable collagen based sponge 501 that has been reduced through rehydration to fold in upon itself to form a sponge that is small enough to be drawn through an implant syringe. FIG. 5C shows an implant syringe 502 having a needle 503, a barrel 504 and a plunger 505. FIG. 5D shows the plunger 505 withdrawn from the barrel 504, creating suction on the reduced sponge 501 wherein the cross-sectional area of the barrel 504 is large enough to allow passage of the reduced sponge 501 into the barrel 504.

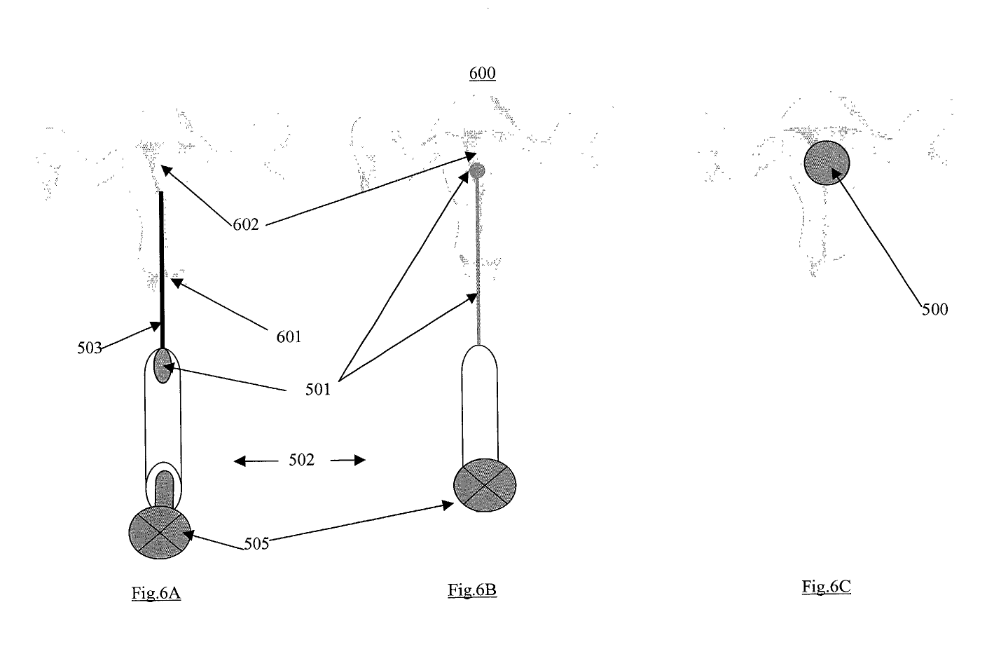

FIG. 6A depicts an implant syringe 502 having a reduced sponge 501 therein, being inserted into a uterus, generally indicated at 600. The implant syringe 502 is positioned such that the needle 503 is passed through the cervix 601 and into the uterine cavity 602. FIG. 6B shows the plunger 505 depressed to force the reduced sponge 501 through the needle 503 and into the uterine cavity 602. FIG. 6C depicts the sponge 500 situated in the uterine cavity 602. As can be seen, the sponge has enlarged through re-hydration to return to its normal size to completely fill the opening of the uterus.

In one embodiment the expandable collagen based sponge implant was made according to the following method. At room temperature tissue collagenous connective tissue was debrided and sectioned into small pieces. Then, 2 L of 0.5N Acetic Acid (Hac) was added to the tissue and the mixture was stirred. After several hours, the mixture was placed into a waring blender and blended until most of the tissue was in suspension. The suspension was then poured through an 850 μm sieve in order to remove undissolved tissue. The collagen was then precipitated by adding NaOH dropwise while stirring until the pH was greater than 5.8, but less than 7.0. After sieving out the solid, the precipitate was resuspended in acetic acid (2 L) by blending. While stirring, NaCl powder was added to the suspension until the salt content was 8% w/v. This caused the collagen to precipitate again. The mixture was sieved and the precipitate was resuspended in 2 L acidic once more by blending. Next. 500 ml of 0.5N acidic acid was added in order to produce a ˜2% collagen suspension. Approximately 100 ml of the suspension was then placed into a dialysis tube and dialyzed for 1 hr against a pH of 7.0 Next, 10 ml of 8% glutaraldehyde solution was added to the buffer to make a 0.16% glutaraldehyde solution. After 3 hours, the glutaraldehyde buffer was replaced with deionized water. The sample was then left overnight, frozen at −40° c. and then lyophilized. The following day, a small section ˜1½ cc was removed from the solidified implant with a scalpel. The sample was rolled and stuffed into an implant syringe. The syringe was fed down the neck of a 10 cc volumetric flask and the plunger depressed to inject the implant. The implant expanded slowly, was easy to compress (˜10 folds or better) and was firm once expanded in water, indicating its applicability to its desired use. Because this material will not break down in situ and induces clot formation as a means to hold it in place, it has utility alone or in combination with one or more agents described above. It will be appreciated in view of the teachings herein that the collagen may be obtained from any available source, e.g., allogenic, autogenic, or xenogenic sources, or synthetic source. Tissues especially useful as a starting material include hard and soft tissues such as, but not limited to, fascia, dermis, ligaments, tendons, bone, pericardium and the like. The subject expanding collagen embodiment has numerous uses such as those described for any other adhesive material described throughout, including but not limited, dermal augmentation, sphincter bulking, urethral blocking, blocking vessels (e.g. arterial-venous malformations, vas deferens, and aneurysms).

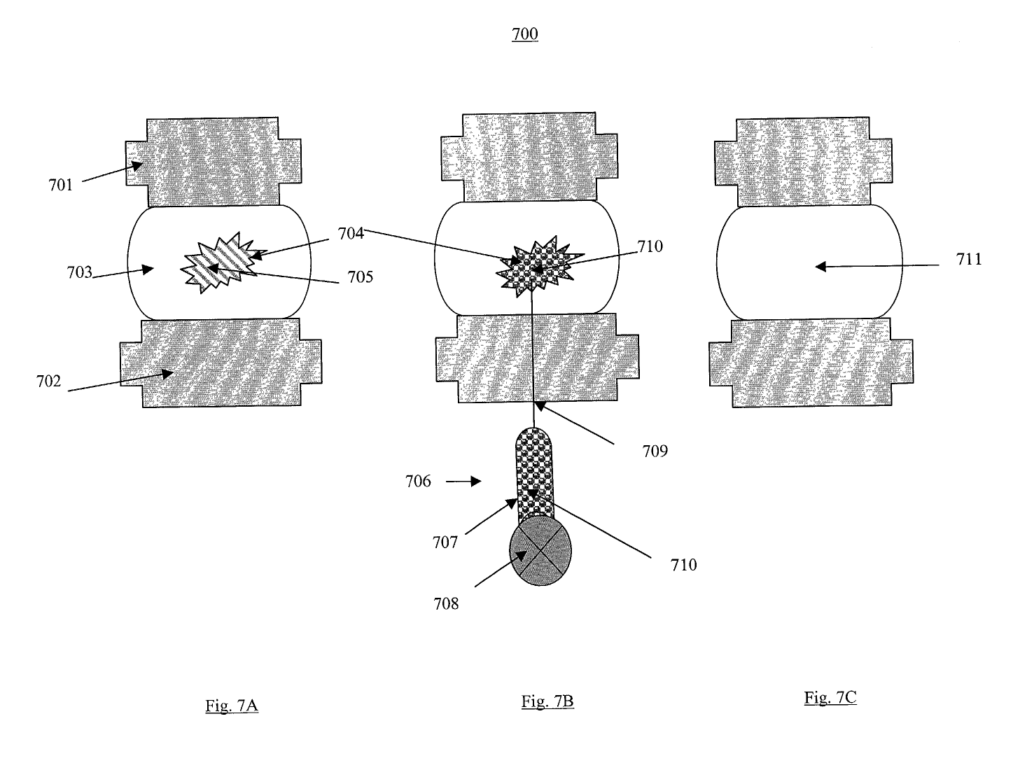

Compositions of the previously described material may be applied to a torn annulus fibrosus to cause an adhesion between tissues. The exact combination of substances may vary so long as the composition, once applied is capable of forming a seal through development of tissue adhesion. FIG. 7A shows an intervertebral disc complex generally indicated at 700, comprising an upper vertebrae 701 a lower vertebrae 702 and a ruptured disc 703. As shown, the disc 703 has ruptured along a portion of the annulus fibrosus 704 which has exposed the nucleus pulposus 705. FIG. 7B depicts disc syringe 706 comprising a barrel 707, a plunger 708 and a needle 709. The barrel is filled with a bioactive composition 710 capable of causing an adhesion in tissue of the annulus fibrosus when applied. As shown the composition 710 is injected into the damaged site of the annulus fibrosus 704 to fill in the area. FIG. 7C shows a intervertebral disc complex after application and assimilation of the bioactive composition showing a repaired disc 711.

Kits of the present invention include 1) an implantable composition comprising a) water-insoluble particles that promote one or more of cellular inflammation, infiltration, or adhesion, and b) a carrier to form a paste or suspension, and 2) instructions for delivering the implantable composition to a lumen, tissue or organ in need of occlusion or bulking, such that upon delivering the implantable composition, an occlusion forms in the lumen, or a bulked up area forms in the tissue or organ of the living mammal. These and other kits also may include specific syringes, needles, catheters, and the like, to be used for delivering the implantable composition.

In particular, kits may include materials for the composition selected from the nonexclusive lists of water-insoluble particles, carriers, growth factors and biologically active agents that are provided in this disclosure.

One embodiment of a kit contains a syringe filled with a predetermined quantity of sized water-insoluble particles, and a second syringe filled with a carrier mixed with a solvent such as water. The syringes are opened and joined together, and the particles and carrier are mixed by alternately moving the plunger of each syringe to move and mix the materials together. Once well mixed, to a desired consistency, a syringe, catheter, or other delivery device is attached to the syringe holding the mixed composition, and the composition is thereby delivered to a lumen, tissue, or organ in need thereof. Another embodiment uses a syringe that has a pliable, flexible section of the syringe barrel. Once all materials are in this syringe, this pliable area is pressed, as by fingers, to mix the materials. The mixed materials, or composition, are then delivered in standard fashion by pressing the plunger of the syringe. These approaches to kits, and reconstituting compositions, is more fully described in co-pending U.S. applications, Ser. Nos. 09/474,276, 09/751,929, and 09/792,894, which are incorporated by reference.

Applications for the use of compositions, methods and kits of the present invention include applying the compositions to block or occlude tear ducts, salivary gland ducts, sweat gland ducts, and arteriovenous connections, to treat conditions where such blockage or occlusion is desired. For example, in a condition known as arteriovenous anastomosis, where an artery and a vein are improperly joined, leading to ‘starving’ of cells that are supplied by a capillary bed that is bypassed due to the anastomosis, the improper junction of the anastomosis may be occluded by use of a composition of the present invention, applied to form a blockage of the improper channel, thereby redirecting the arterial flow to the capillary bed.

Still other applications use compositions, methods and kits of the present invention to bulk up sphincter muscles and vocal chords. In these applications it is preferred that the particulate component of the composition does not resorb into the body, e.g. crystalline hydroxyapatite.

Another embodiment of the present invention is to cut of the blood supply to a tumor by occluding an artery, and/or a capillary plexus, that directly supplies the tumor.

Another embodiment of the present invention is to form a blockage or occlusion in manmade channels made in bones such as the skull. For instance, a temporary cranial tap may be made by a surgeon to release blood that has pooled between the brain and the skull, such as due to a concussion. A composition of the present invention may be used to fill such a channel after the release of the blood and pressure. This prevents the passage of extra cranial fluids, or pathogens, through the channel.

The compositions, methods and kits of the present invention may be used for the blockage or occlusion of other ducts, channels, and lumens, and the bulking up of tissues and muscles other than those described above, such as may be envisioned and practiced by one of skill in the art.

Preparations of a Preferred Type of Composition

The terms “thermoplastic,” “thermally cross-linked” or “thermally cross-linkable” are used herein to describe the property of a composition which contains molecules which, at or below a given temperature and concentration, associate in such a fashion as to result in gelation of a solution containing these molecules.

The gelatin acts as a carrier phase and has the ability to thermally cross-link over a very small temperature range. This thermal cross-linking reaction is largely controlled by physical entanglement and hydrogen bonding between chains, and so is dependant on concentration and temperature. Additionally, since gelatin has been used extensively in the medical market, its in vivo properties are thoroughly studied. The gel-foam sponge is the most familiar application of this biopolymer. Studies have indicated that gelatin is only mildly antigenic upon implantation, and is comparable in some of its properties to collagen.

The manufacture of gelatin is based on the partial hydrolysis of collagen. Collagen is available from skin, bone, cartilage, tendon and other connective tissue. Skin and bone yield Type I and Type III collagen molecules, while tendon yields nearly pure Type I collagen, and cartilage yields a mixture of Type II and rarer types of collagen molecules. Gelatin molecules resemble collagen triple helices, however, they are partially hydrolyzed. As a result, in solution they have little organization. But, as the solution cools, the gelatin molecules begin to form helical structures. As the solution cools further, the viscosity increases and a phase transformation from a solution to a gel occurs. This phase change is reversible when heat is added.

The set time and set temperature of a gelatin solution are dependent on the concentration of gelatin in solution, the molecular weight, or intrinsic viscosity, of the gelatin molecules, and the pH of the solution. At the isoelectric point, or the pH at which the gelatin molecules are electrically neutral, the set time is the shortest.

Collagen can be partially hydrolyzed by several methods. The Type A process is the simplest and most rapid process, in which dilute acid (e.g. less than 1 M HCl) is used to partially hydrolyze the collagen. Type A processing is generally used with porcine skin and demineralized bovine bone. The Type B process uses an alkaline solution to partially hydrolyze the collagen. Type B processing is generally used with bovine hide and demineralized bovine bone. Finally, enzymes, such as pepsin, may be used to partially hydrolyze collagen. Pepsin preferentially cleaves peptide bonds between aromatic amino acids. Pepsin also acts as an esterase, but amides of amino acids are not hydrolyzed.

As one example of this method, the gelatin is prepared from the bones of the species into which the compositions are to be implanted, by crushing and defatting the bones followed by soaking for about 24 hours in approximately 300 mg/L pepsin in a 0.5 M acetic acid at 33° C. The pH of the resulting solution is brought to 9.0 with sodium hydroxide to denature the pepsin, then it is returned to 7.0 with hydrochloric acid. The temperature of the solution is raised to 60° C. for about 15 to 30 minutes and returned to 4° C. to effect denaturation of remaining collagen and complete conversion to gelatin. The resulting solution is filtered to remove particulates and dialyzed against distilled water for 48 hours in a 50K-100K molecular weight cut-off (50K-100K MWCO) dialysis membrane. After lyophilization, the gelatin is redissolved in phosphate buffered saline (PBS) or water to an effective concentration of about 30-45 weight percent of gelatin in solution.

The gelatin content of the composition is desirably between about 15-30% (w/w). The gelatin may be derived from the same or different species than that into which the composition is to be implanted. For example, human, porcine, bovine, equine, or canine gelatin is derived from collagen sources such as bone, skin, tendons, or cartilage, and may then be mixed with non-osteoinductive DBM. As noted above, the collagen is converted to gelatin via, liming, acidification or by enzymatic extraction, for example by pepsin or like enzymatic treatment, followed by denaturation by heat or other means. The gelatin may be derived from tissue by mastication of the tissue, followed by an extended treatment capable of breaking cross-links in the long collagen chains. In one embodiment, the tissue is ground then soaked for about 24-72 hours at between about 240° C. in dilute acid, such as 0.1 normal acetic acid. Preferably, an enzyme such as pepsin at a sufficiently high concentration is added. Pepsin concentrations of between about 10-20,000 i.u./liter, 100-2,000 i.u/liter, or like concentrations are added to the dilute acid at the start of the treatment, with the period of treatment being adjusted according to the enzyme concentration used. Solids are removed from the composition, for example by centrifugation, and the supernatant material in solution having a molecular weight of about 50,000 daltons or higher is retained. This may be achieved by any of a number of methods known in the art including, but not limited to, dialyzing the supernatant in a 50,000 dalton molecular weight cut-off membrane against several changes of solution, ultrafiltration against a membrane having a like molecular weight cut-off, (MWCO) or gel permeation chromatography through a medium having a 50,000 dalton molecular mass cut-off. It will be recognized by those skilled in the art that the higher the MWCO of the gelatin, the lower the yield. Accordingly, lower MWCO gelatin preparations, down to about 1000 dalton MWCO's could be used, recognizing that undesirable low molecular weight species might thereby be retained.

The gelatin solution resulting from the foregoing extraction is preferably denatured, for example by heat-treatment to above about 50° C. The denatured protein is then stored in a frozen state or it may be freeze-dried or precipitated, for example in a volatile organic solvent, and reconstituted in a solution, such as an isotonic saline solution, at a concentration of between about 15-30% (w/w) gelatin.

A partially hydrolyzed collagen is a preferred carrier material. In one example, dermis particles (or DBM or tendon particles, or a combination thereof) are simply heated to 80-100 degrees Celsius in water for one hour. The resulting mixture is then freeze-dried to form a gelatin-like substance.

The non-osteoconductive demineralized bone is preferably in a powdered form, and is preferably composed of particles in the size range between about 1-70 μm particle size range. Methods for producing demineralized bone powder are known in the art (see for example U.S. Pat. No. 5,405,390, herein incorporated by reference for this purpose), and are not, therefore, elaborated here. Demineralized bone powder is mixed with the gelatin solution prepared as described above, to form a composition comprising about 0-25% (w/w) demineralized bone powder.

Compositions prepared as described above are easily extruded from a syringe, particularly when the temperature is elevated to above about 40° C., for example by immersion in a water bath, by limited treatment in a microwave, by placement in a syringe warmer, or any of a number of other methods for heating the container. The extruded gel is resilient, sticky and easily forms into a bolus inside the lumen. The composition retains its strength and is poorly soluble in saline once it sets-up.

Accordingly, having generally described the compositions of this invention, and taking into account the specifics of the exemplary support provided below, the following guidelines for the preparation and use of the composition of this invention are provided:

It is noted that the figures depicting this invention is merely representative of particular embodiments and are not meant to limit the range of possible configurations to which this invention may be applied. The features are represented and described by numbers consistent from drawing to drawing, where possible.

Having generally described this invention, including the best mode thereof, those skilled in the art will appreciate that the present invention contemplates the following embodiments, and equivalents thereof. However, those skilled in the art will appreciate that the scope of this invention should be measured by the claims appended hereto, and not merely by the specific embodiments exemplified herein. Furthermore, the teachings of all cited references are incorporated by reference to the extent they are not inconsistent with the teachings herein.

What is claimed is:

1. A method of administering a closure-forming or bulking up composition in a living mammal, comprising
a. mixing together, to form a composition, at least one type of water-insoluble particle and a carrier; and
b. applying the composition to at least one specific area of a lumen or other body region in need of closure;
whereby applying the composition results in closure or bulking up of the at least one specific area.

2. A method of administering a closure-forming or bulking up composition in a living mammal, comprising
a. mixing together, to form a composition, at least one type of water-insoluble particle that promotes responsive body processes and a carrier; and
b. applying the composition to at least one specific area of a lumen or other body region in need of closure;
whereby applying the composition results in closure or bulking up of the at least one specific area.

3. A method of administering a closure-forming or bulking up composition in a living mammal, comprising
a. obtaining at least one type of water-insoluble particle that promotes responsive body processes, from the group consisting fine particles of bone; fine particles of hydroxyapatite, in the 1 to 70 micrometers particle size range; non-osteoinductive demineralized bone matrix, preferably in the 125-250 micrometer particle size range; collagen shards, preferably in the 125-250 micrometer particle size range; insoluble salts, and talc;
b. mixing together, to form a composition, said at least one type of water-insoluble particle and a carrier; and
c. applying the composition to at least one specific area of a lumen or other body region in need of closure, or tissue or organ in need of bulking up;
whereby applying the composition results in closure or bulking up of the at least one specific area.

4. The method according to claim 3, wherein said carrier is selected from the group consisting of: collagen; gelatin; carboxymethyl cellulose; glycosaminoglycans; proteoglycans, polyvinyl alcohol; thrombin; fibrin; albumin; aphiphillic derivatives of sodium alginate; chitosan, polyalcohols, polyamines, polyvinyls, polyamides, polyesters, polyanhydrides, polyortho-esters, polyurethanes, polycarbonates, polyphosphazines, polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and mixtures thereof.

5. The method according to claim 3, additionally comprising the step of including an additive in the carrier, said additive selected from the group consisting of growth factors, biologically active agents, and combinations thereof.

6. The method according to claim 5, wherein at least one growth factor is selected from the group consisting of PDGF, FGF, VEGF, BMP, EGF, ECGF, and PDGF.

7. The method according to claim 5, wherein said biologically active agent is selected from the group comprising hyaluronic acid, chondroitan sulfate, keratin sulfate, dermatan sulfate, heparin, heparin sulfate, galactosaminoglycuronoglycan sulfate, proteoglycans; members of the Selectin, IgSF, Integrin, or Cadherin superfamilies; laminin, entactin, nidogen, recombinant osteogenic protein-1 and combinations thereof.

8. The method of claim 3 wherein said applying of the composition is done by percutaneous injection to a location in need thereof.

9. An implantable composition for administration to a lumen of a living mammal, to close the lumen, comprising:
a. water-insoluble particles, and
b. a carrier compound, such that when combined in a liquid, the water-insoluble particles are suspended in a solution.

10. An implantable composition for administration to a lumen of a living mammal, to close the lumen, comprising:
a. water-insoluble particles that promote responsive body processes, and
b. a carrier compound, such that when combined in a liquid, the water-insoluble particles are suspended in a solution.

11. An implantable composition for administration to a lumen of a living mammal, to close the lumen, comprising:
a. water-insoluble particles that promote responsive body processes, and
b. a carrier compound, such that when combined in a liquid, the water-insoluble particles are suspended in a solution,
wherein said carrier compound is thermoplastic gelatin and said solution is flowable at a temperature above the body temperature of a subject living mammal, and is not flowable at the said body temperature.

12. The implantable composition according to claim 9, wherein the carrier is selected from the group consisting of: collagen; gelatin; carboxymethyl cellulose; glycosaminoglycans; proteoglycans, polyvinyl alcohol; thrombin; fibrin; albumin; aphiphillic derivatives of sodium alginate; chitosan, polyalcohols, polyamines, polyvinyls, polyamides, polyesters, polyanhydrides, polyortho-esters, polyurethanes, polycarbonates, polyphosphazines, polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and mixtures thereof.

13. The implantable composition according to claim 11, wherein the water-insoluble particles are selected from the group consisting of fine particles of bone; fine particles of hydroxyapatite, in the 1 to 70 micrometers particle size range; non-osteoinductive demineralized bone matrix, preferably in the 125-250 micrometer particle size range; collagen shards, preferably in the 125-250 micrometer particle size range; insoluble salts, and talc.

14. An implantable composition for administration to a tissue or organ of a living mammal, to add bulk to the tissue or organ, comprising water-insoluble particles in a carrier compound, such that when combined in a liquid, the water-insoluble particles are suspended in a solution.

15. An implantable composition for administration to a tissue or organ of a living mammal, to add bulk to the tissue or organ, comprising:
a. water-insoluble particles that promote responsive body processes, and
b. a carrier compound, such that when combined in a liquid, the water-insoluble particles are suspended in a solution.

16. An implantable composition for administration to a tissue or organ of a living mammal, to add bulk to the tissue or organ, comprising:
a. water-insoluble particles that promote responsive body processes, and
b. a carrier compound, such that when combined in a liquid, the water-insoluble particles are suspended in a solution,
wherein said carrier compound is thermoplastic gelatin and said solution is flowable at a temperature above the body temperature of a subject living mammal, and is not flowable at the said body temperature.

17. The implantable composition according to claim 14, wherein the carrier is selected from the group consisting of: collagen; gelatin; carboxymethyl cellulose; glycosaminoglycans; proteoglycans, polyvinyl alcohol; thrombin; fibrin; albumin; aphiphillic derivatives of sodium alginate; chitosan, polyalcohols, polyamines, polyvinyls, polyamides, polyesters, polyanhydrides, polyortho-esters, polyurethanes, polycarbonates, polyphosphazines, polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and mixtures thereof.

18. The implant according to claim 16, wherein the water-insoluble particles are selected from the group consisting of fine particles of bone; fine particles of hydroxyapatite, in the 1 to 70 micrometers particle size range; non-osteoinductive demineralized bone matrix, preferably in the 125-250 micrometer particle size range; collagen shards, preferably in the 125-250 micrometer particle size range; insoluble salts, and talc.

19. A method of promoting the formation of an occlusion in a lumen, or a bulked-up region in a tissue or organ, comprising the steps of:
a. preparing a composition comprising water insoluble particles, said particles promoting formation of an adhesion, and
b. injecting a quantity of said composition into a body area of a living mammal, such that adhesion formation is promoted in the area of the injecting.

20. The method according to claim 18, wherein the injection is percutaneous.

21. A method of promoting the lessening or cessation of menorrhea, comprising the steps of:
a. preparing a composition comprising water insoluble particles, said particles promoting an inflammatory response, and
b. applying a quantity of said composition into the uterus of a living female mammal in need of said lessening or cessation of menorrhea,
whereby said inflammatory response results in said lessening or cessation of menorrhea.

22. The method according to claim 20, wherein said applying is through a catheter passing through the cervix.

23. The method according to claim 3, wherein said closure is for a lumen or channel in a structure selected from the group consisting of a vas deferens duct, a tear duct, a salivary gland duct, a sweat gland duct, an arteriovenous connection, an arteriovenous anastomosis, an artery supplying a tumor, a capillary plexus supplying a tumor, or a manmade channel in need of said closure.

24. The method according to claim 3, wherein said bulking up is for a tissue or organ selected from the group consisting of sphincter muscles and vocal chords

25. The implantable composition according to claim 9, wherein said lumen is selected from the group consisting of a vas deferens duct, a tear duct, a salivary gland duct, a sweat gland duct, an arteriovenous connection, an arteriovenous anastomosis, an artery supplying a tumor, a capillary plexus supplying a tumor, or a man-made channel in need of said closure.

26. The method according to claim 14, wherein said administration to add bulk is to a tissue or organ selected from the group consisting of sphincter muscles and vocal chords.

27. The implantable composition according to claim 18, wherein said adhesion formation is in a lumen selected from the group consisting of a vas deferens duct, a tear duct, a salivary gland duct, a sweat gland duct, an arteriovenous connection, an arteriovenous anastomosis, an artery supplying a tumor, a capillary plexus supplying a tumor, or a manmade channel in need of said closure.

28. The method according to claim 18, wherein said adhesion formation is in a tissue or organ selected from the group consisting of sphincter muscles and vocal chords.

29. A kit for lumen-closing or a tissue-bulking implant for a living mammal, comprising:
a. an implantable composition comprising water-insoluble particles that promote one or more of cellular inflammation, infiltration, or adhesion, and a carrier to form a paste or suspension; and
b. instructions for delivering said implantable composition to a lumen, tissue or organ in need of occlusion or bulking,
wherein upon said delivering, an occlusion forms in the lumen, or a bulked up area forms in the tissue or organ of the living mammal.

30. The kit according to claim 28, wherein said carrier is selected from the group consisting of: collagen; gelatin; carboxymethyl cellulose; glycosaminoglycans; proteoglycans, polyvinyl alcohol; thrombin; fibrin; albumin; aphiphillic derivatives of sodium alginate; chitosan, polyalcohols, polyamines, polyvinyls, polyamides, polyesters, polyanhydrides, polyortho-esters, polyurethanes, polycarbonates, polyphosphazines, polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and mixtures thereof.

31. The kit according to claim 28, wherein said water-insoluble particles are selected from the group consisting of fine particles of bone; fine particles of hydroxyapatite, in the 1 to 70 micrometers particle size range; non-osteoinductive demineralized bone matrix, preferably in the 125-250 micrometer particle size range; collagen shards, preferably in the 125-250 micrometer particle size range; insoluble salts, and talc.

32. The kit according to claim 28, wherein said instructions provide for mixing said water-insoluble particles and said carrier in a syringe.

33. The kit according to claim 31, wherein said syringe has a flexible area of its barrel to facilitate mixing.

34. The kit according to claim 28, wherein at least one of said water insoluble particles and said carrier is provided in a syringe.

35. A method for closure of a lumen, or bulking up of a tissue or organ in a living mammal, comprising:
a. obtaining a kit comprising an implantable composition comprising water-insoluble particles that promote one or more of cellular inflammation, infiltration, or adhesion, and a carrier to form a paste or suspension; and
b. administering said implantable composition to a lumen in need of closure, or to a tissue or organ in need of bulking up.

36. The method according to claim 34, additionally comprising following prescribed instructions for the preparation and mixing of said water-insoluble particles and said carrier to form said implantable composition.

37. The method according to claim 34, wherein said carrier is selected from the group consisting of: collagen; gelatin; carboxymethyl cellulose; glycosaminoglycans; proteoglycans, polyvinyl alcohol; thrombin; fibrin; albumin; aphiphillic derivatives of sodium alginate; chitosan, polyalcohols, polyamines, polyvinyls, polyamides, polyesters, polyanhydrides, polyortho-esters, polyurethanes, polycarbonates, polyphosphazines, polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and mixtures thereof.

38. The method according to claim 34, wherein said, wherein said water-insoluble particles are selected from the group consisting of fine particles of bone; fine particles of hydroxyapatite, in the 1 to 70 micrometers particle size range; non-osteoinductive demineralized bone matrix, preferably in the 125-250 micrometer particle size range; collagen shards, preferably in the 125-250 micrometer particle size range; insoluble salts, and talc.

39. A method of promoting the lessening or cessation of menorrhea, comprising the steps of:
a) constructing an expandable sponge; and
b) implanting said sponge into the uterine cavity of a patient.

40. The method of claim 39, wherein said sponge is comprised of collagen, gelatin, carboxymethylcellulose, hyaluronic acid, or combinations thereof.

41. The method of claim 40, wherein said collagen is cross-linked.

42. The method of claim 39, wherein said sponge is dehydrated and compressed to fit inside a syringe.

43. The method of claim 39 wherein said dehydrated sponge is injected into a lumen or cavity.

44. The method of claim 43, wherein said dehydrated sponge is rehydrated in situ to expand to normal size.

45. The method of claim 39, wherein said implant is held in place through coagulation of blood surrounding said implant.

46. An expandable tissue based sponge for implantation into a lumen.

47. The expandable tissue based sponge of claim 46, wherein said tissue is cross-linked collagen.

48. The expandable tissue based sponge of claim 46, wherein said sponge is injected into a lumen or cavity to form an occlusion.

49. The expandable tissue based sponge of claim 48, wherein said cavity is a uterine cavity.

50. The expandable sponge of claim 45, where said sponge promotes Asherman's syndrome when implanted into a uterus.

51. A method of repairing a ruptured intervertebral disc comprising administering a closure-forming composition to an annulus fibrosus.

52. The method of claim 51, wherein said method further comprises
a. mixing together, to form a composition, at least one type of biologically active agent and a carrier; and
b. applying the composition to at least one specific area of a ruptured in need of closure; whereby applying the composition results in closure of the at least one specific area.

53. The method according to claim 52, wherein said biologically active agent is selected from the group comprising hyaluronic acid, chondroitan sulfate, keratin sulfate, dermatan sulfate, heparin, heparin sulfate, galactosaminoglycuronoglycan sulfate, proteoglycans; members of the Selectin, IgSF, Integrin, or Cadherin superfamilies; laminin, entactin, nidogen, recombinant osteogenic protein-1 and combinations thereof.

54. The method according to claim 52, wherein said carrier is selected from the group consisting of: collagen; gelatin; carboxymethyl cellulose; glycosaminoglycans; proteoglycans, polyvinyl alcohol; thrombin; fibrin; albumin; aphiphillic derivatives of sodium alginate; chitosan, polyalcohols, polyamines, polyvinyls, polyamides, polyesters, polyanhydrides, polyortho-esters, polyurethanes, polycarbonates, polyphosphazines, polysilicates, Zyderm™, Zyplast™, Fibrel™, Dermologen™, Micronized Alloderm™, Isologen™, medical grade silicone, Bioplastique™, Arteplast™, Artecoll™, Formacryl™, hydrogels, ePTFE, CoSeal and mixtures thereof.

55. A method of blocking or filling at least one lumen comprising implanting an expandable sponge or hydrogel into said at least one lumen.

56. The method of claim 55, wherein said lumen is a blood vessel or vas deferens.

57. The method of claim 55, further comprising loading said expandable sponge or hydrogel into a syringe and delivering said expandable sponge or hydrogel to an intended site.
